(19) United States
(12) Patent Application Publication
(10) Pub. No.: US 2010/0166692 A1
(43) Pub. Date: Jul. 1, 2010

(54) METHOD FOR TREATING DAMAGED HAIR IN CONJUNCTION WITH THE RELAXING PROCESS

(76) Inventors: Yin Z. Hessefort, Naperville, IL (US); Brian T. Holland, Madison, WI (US); Jeffery M. Atkins, Aurora, IL (US); Sascha Weiz, Chicago, IL (US); Xiaojin Harry Li, Bartlett, IL (US); Virginie Kompalitch, Paris (FR)

Correspondence Address: NALCO COMPANY, 1601 W. DIEHL ROAD, NAPERVILLE, IL, 60563-1198, US

(21) Appl. No.: 12/346,310

(22) Filed: Dec. 30, 2008

(51) Int. Cl. A61K 8/81; A61Q 5/04

(52) U.S. Cl. 424/70.2

(57) ABSTRACT

A method of treating one or more damaged hair shafts, each hair shaft including a cuticle layer and a cortex enclosed in the cuticle layer is disclosed. The method comprises: selecting one or more polymers that can penetrate the hair shafts with a pore size of about 5 angstroms to about 5000 angstroms; and treating the hair shafts by applying an effective amount of a composition containing said anionic polymers to said hair shafts.

### BACKGROUND OF THE INVENTION

Following either popular or celebrity fashion trends, more and more consumers use hair treatments to pursue fashionable hairstyles. The treatments vary greatly but the relaxing treatment is one of the harshest to hair. Although this hairstyle techniques greatly satisfy consumers' needs, they also cause severe hair damage, especially when the treatments are used repetitively. Moreover, various daily actions to the hair, for example hair brushing, hair blow-drying, and sun light exposure add more damage to the hair.

It is generally accepted that chemical treatment and/or UV exposure causes hair damage, which results in increased porosity and swelling of the hair cuticle. That is why hair becomes rough, coarse and dull when damage happens to the hair. Furthermore, hair looses its tensile strength when damage occurs in the hair's cortex, since the cortex is believed to be primarily responsible for the tensile properties of human hair. The cuticle of the hair is an important factor in torsional mechanical properties, but its contribution to bulk longitudinal mechanical strength is minor. Therefore, the measurement of tensile strength not only is an evaluation method of hair damage, but also an indication to determine if damage has penetrated to the cortex. One of the ways to restore natural quality of damaged hair is to recover its reduced tensile strength.

A method of treating hair that addresses at least some of the above-mentioned problems is therefore desired.

### SUMMARY OF THE INVENTION

The present disclosure provides for a method of treating one or more hair shafts, each hair shaft including a cuticle layer and a cortex enclosed in the cuticle layer comprising: selecting one or more polymers that can penetrate the hair shafts with a pore size of about 5 angstroms to about 5,000 angstroms; and treating the hair shafts by applying an effective amount of a composition containing said anionic polymers or copolymers to said hair shafts.

### DETAILED DESCRIPTION OF THE INVENTION

One or more hair shafts are treated with one or more polymers that can penetrate a hair shaft with a pore size of about 5 angstroms to about 5000 angstroms.

In one embodiment, the hair shaft pore size is between about 10 angstroms and about 1000 angstroms.

In another embodiment, the purpose of the treatment is to nourish and/or repair the hair shaft.

In another embodiment, the purpose of the treatment is to improve the tensile strength of the hair.

Generally, the polymers utilized should be of sufficient size to penetrate into the cortex of the hair shaft, but not easily migrate out of the cortex. One of ordinary skill in the art could determine whether a polymer meets this particularly criteria without undue experimentation. Therefore, polymers that are linear, branched, hyperbranched, or dendritic may meet this criteria.

Various types and conformations of polymers may be utilized to treat a hair shaft.

In one embodiment, the polymers are selected from the groups consisting of homopolymers, copolymers, terpolymers, and a combination thereof.

In another embodiment, the polymers are selected from the group consisting of cationic polymers (CIP2), anionic polymers (CIP1), non-ionic polymers, amphoteric polymers, zwitterionic polymers, and a combination thereof.

In another embodiment, the polymers are linear. One of ordinary skill in the art would know the scope of the term linear polymer, however, in the present case, that definition can be expanded to include a polymer that is arranged in a chainlike fashion with few branches or bridges or cross-links between the chains.

In another embodiment, the polymers have a weight average molecular weight of from about 300 daltons to about 80,000 daltons, excluding PolyDADMAC wherein the upper limit of said range for PolyDADMAC is less than 15,000 daltons.

In its principal aspect, this invention is directed to a cosmetically acceptable hair repairing polymer whose composition comprising from about 0.1 to about 10 weight percent, based on polymer solids, of an anionic polymer, wherein the anionic polymer is composed of homopolymer of polyacrylic acid or copolymer from about 10 to about 90 mole percent of polyacrylic acid or a base addition salt thereof and from about 90 to about 20 mole percent of one or more anionic or nonionic monomers.

“Anionic monomer” means a monomer as defined herein which possesses a net negative charge above a certain pH value. Representative anionic monomers include base addition salts of acrylic acid, methacrylic acid, itaconic acid, 2-acrylamido-2-methyl propane sulfonic acid, sulfopropyl acrylate or methacrylate or other water-soluble forms of these or other polymerizable carboxylic or sulfonic acids, sulphomethylated acrylamide, allyl sulphonate, sodium vinyl sulphonate, and the like. Preferred anionic monomers are acrylic acid and 2-acrylamido-2-methyl propane sulfonic acid.

“Base addition salt” means the salt resulting from reaction of a carboyxlic acid (—CO₂H) group with a suitable base such as the hydroxide, carbonate, or bicarbonate of a metal cation or tetraalkylammonium cation, or with ammonia, or an organic primary, secondary, or tertiary amine of sufficient basicity to form a salt with the carboxylic acid group. Representative alkali or alkaline earth metal salts include sodium, lithium, potassium, calcium, magnesium, and the like. Representative organic amines useful for the formation of base addition salts include, ethylamine, diethylamine, ethylenediamine, ethanolamine, diethanolamine, piperazine, and the like. Preferred base addition salts include the sodium and ammonium salts.

Specifically, poly(sodium acrylate) has a weight average molecular weight of about 3,000 daltons to about 15,000 daltons for the treatment of bleaching damaged hair to improve hair tensile strength.

More specifically, copolymer of acrylic acid and 2-acrylamido-2-methyl-1-propanesulfonic acid or a base addition salt with molecular weight of about 1000 daltons to about 12000 daltons added to a commercial relaxer base to prevent hair from relaxer damage.

The composition containing the polymers may be in various forms. One of ordinary skill in the art would know how to formulate the polymers with cosmetically acceptable excipients and/or other components of a composition.

The following example is not meant to be limiting.

### EXAMPLES

For this EXAMPLE section, the weight-average molecular weight of polymer was determined by a size-exclusion chromatography/multi-angle laser light scattering (or SEC/MALLS) technique. Size exclusion chromatography (SEC) was performed by using a series of TSK-GEL PW columns from TOSOH BIOSCIENCE, a multi-angle laser light scattering detector (MALLS, model: DAWN DSP-F) and an interferometric refractometer (OPTILAP DSP) from Wyatt Technology. Data collection and analysis were performed with ASTRA software from Wyatt Technology.

a. Tensile Strength Measurements

A tensile strength test was done on chemically damaged hair. The protocol included the following steps.

Virgin brown hair was bleached by immersion in 6% hydrogen peroxide solution containing 1.7% ammonium hydroxide and 10% urea at 40±1° C. for 15 minutes. The bleached hair was then treated in 1% (solid) polymer solution for 5 minutes and rinsed under deionized water for 10 seconds.

The diameter of forty hair strands was randomly selected from each treated and untreated (“control”) testing group were measured using a Fiber Dimensional Analysis System (Mitutoyo, Model LSM 5000). The hair samples were placed in a DiaStron Miniature Tensile Tester (Model 170/670) for the determination of tensile strength in a wet condition. The total work force normalized with hair diameter was calculated by using DiaStron software (MTTWIN Application Software Version 5.0). The mean values obtained from 40 hair strands were analyzed using Tukey HSD statistical analysis to compare all the testing pairs (ANOVA one-way analysis of variance from JMP statistical software, SAS Institute, Cary, N.C., U.S). The testing results and statistical analysis are summarized in following tables.

b. Method of Treatment Using Commercial Relaxer

Relaxer with Polymer means anionic polymer was added to a commercial relaxer at 0.5% (solid) level. Control means relaxer without polymer.

“Initial” means the tensile strength was tested right after polymer was added to the relaxer. “After” means the tensile strength was tested after relaxer containing polymer went through 3 months stability test at 45° C. temperature.

Wear gloves and slowly apply commercial relaxer (Sofn'free/Cortical cream relaxer/Super for Coarse Hair, M&M Products) in 1 to 2 weight ratio of hair to relaxer onto hair tress. Cover the treated hair tress with aluminum foil for 15 minutes. Rinse hair thoroughly with warm water until all the relaxer is removed. Let hair air-dried and test the tensile strength.

TABLE 1
Chemistry and Molecular Weight of the Anionic Polymer

| | Molecular | |
|---|---|---|
| Name | Weight | Chemistry |
| Anionic Polymer I | 10000 | Poly(sodium acrylate) |

TABLE 2
Tensile Strength Measurement for the Treatment Listed in Table 1

| | Tensile | |
|---|---|---|
| Sample Name | Strength (J) | % Improvement |
| Control | 0.000955 | |
| Anionic Polymer I | 0.00115 | 20.42 |

TABLE 3
Chemistry and Molecular Weight of the Anionic Copolymer

| | Molecular | |
|---|---|---|
| Name | Weight | Chemistry |
| Anionic | 12000 | Poly(acrylic acid/ |
| Copolymer II | | acrylamidomethyl propane |
| | | sulfonate |

TABLE 4
Tensile Strength Measurement for the Treatment Listed in Table 3

| | |
|---|---|
| Tensile | |
| Strength | % |
| (mJ) | Improvement |

| Sample Name | Initial | After | Initial | After |
|---|---|---|---|---|
| Control | 0.81 | 0.86 | | |
| Anionic Copolymer II | 1.19 | 1.25 | 47 | 45 |

c. Surface Area Measurements

Surface area analysis was also done both on treated and untreated hair tresses to understand if low molecular weight polymer species penetrated the hair shaft. The protocol included the following steps.

Surface area analysis was carried out via a nitrogen adsorption analysis. Nitrogen adsorption analyses on hair samples were conducted using a Quantachrome Autosorb-1C instrument Samples were cut to very fine pieces and then added to a sample cell where they were placed under vacuum at 145° C. for 0.5 hours. Complete water removal is necessary to obtain accurate measurements, which is why 145° C. was used. This value is based on the data collected from Differential Scanning Calorimetry (D)SC) in which dehydration peak appears at around 125° C. A 5-pt BET (Brunauer-Emmett-Teller) surface area analysis was used for all samples. The decrease of surface area indicates that the low molecular weight polymers penetrated the hair and took up the pore spaces, which are distributed throughout the hair shaft.

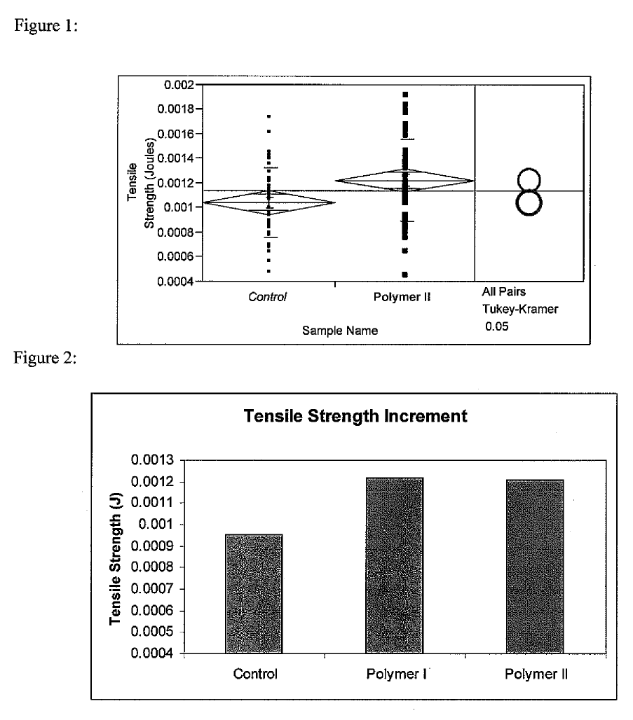

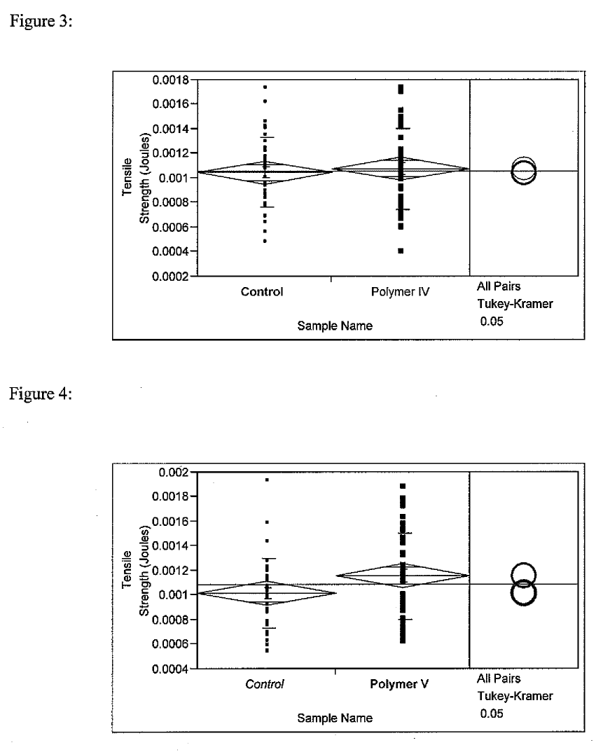

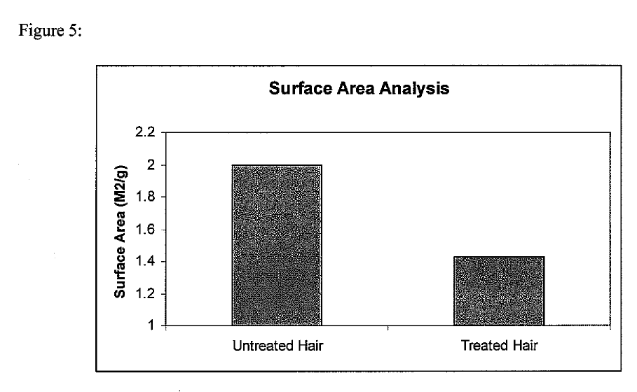

The results for the surface analysis study are illustrated in FIG. 5. Gas sorption analysis from FIG. 5 shows the significant decrease in surface area of hair shafts treated with Polymer II, which illustrates the effective penetration of low molecular weight polymers into the hair shafts.

We claim:

1. A method of treating one or more hair shafts which has or will have damage a hair relaxer treatment, each hair shaft including a cuticle layer and a cortex enclosed in the cuticle layer comprising: selecting one or more anionic polymers that can penetrate the hair shafts with a pore size of about 5 angstroms to about 5000 angstroms; and treating the hair shafts by applying an effective amount of a composition containing said anionic polymers to said hair shafts.

2. The method of claim 1 wherein said anionic polymers have a weight average molecular weight of from about 300 daltons to about 80,000 daltons.

3. The method of claim 1 wherein said anionic polymers contain one or more anionic monomers selected from the group consisting of anionic monomers include base addition salts of acrylic acid, methacrylic acid, itaconic acid, 2-acrylamido-2-methyl propane sulfonic acid, sulfopropyl acrylate or methacrylate or other water-soluble forms of these or other polymerizable carboxylic or sulfonic acids, sulphomethylated acrylamide, allyl sulphonate, sodium vinyl sulphonate, and the like. Preferred anionic monomers are acrylic acid and 2-acrylamido-2-methyl propane sulfonic acid.

4. The method of claim 1 wherein said anionic polymers are selected from the groups consisting of homopolymers, copolymers, and terpolymers, and a combination thereof.

5. The method of claim 1 wherein said anionic polymers are linear or chainlike fashion with few branches or bridges or crosslinks between the chains.

6. The method of claim 1 wherein said composition containing the anionic polymers is applied to the hair prior to the application of a relaxing treatment.

7. The method of claim 1 wherein said composition containing the anionic polymers is applied to the hair simultaneous to the application of a relaxing treatment.

8. The method of claim 1 wherein said composition containing the anionic polymers is applied to the hair after the application of a relaxing treatment.

9. The method of claim 1 wherein said anionic polymer has a low molecular weight.

10. The method of claim 9 wherein said anionic polymer has a low molecular weight of about 300 daltons to about 10,000 daltons.

11. The method of claim 9 wherein said anionic polymer has a low molecular weight of about 300 daltons to about 12,000 daltons.

12. The method of claim 1 wherein said composition contains one or more cosmetically acceptable excipients.

13. The method of claim 1 wherein said excipients are selected from the group consisting of water, saccharides, surface active agents, humectants, petroleum, mineral oil, fatty alcohols, fatty ester emollients, waxes and silicone-containing waxes, silicone oil, silicone fluid, silicone surfactants, volatile hydrocarbon oils, quaternary nitrogen compounds, amine functionalized silicones, conditioning polymers, rheology modifiers, antioxidants, sunscreen active agents, di-long chain amines from about C₁₀ to C₂₂, long chain fatty amines from about C₁₀ to C₂₂, fatty alcohols, ethoxylated fatty alcohols and di-tail phospholipids.

14. The method of claim 1 wherein said polymer is Polyacrylic acid acrylamidomethyl propane sulfonate and/or its salt.

15. The method of claim 1 wherein said pore size is from about 5 angstroms to about 5,000 angstroms.
